(19) United States
(12) Patent Application Publication
(10) Pub. No.: US 2025/0295367 A1
(43) Pub. Date: Sep. 25, 2025

(54) IMAGE PROCESSING DEVICE, IMAGE PROCESSING METHOD, AND IMAGE PROCESSING PROGRAM

(71) Applicant: FUJIFILM CORPORATION, Tokyo (JP)

(72) Inventor: Tatsuki KOIKE, Tokyo (JP)

(21) Appl. No.: 19/064,744

(22) Filed: Feb. 27, 2025

(30) Foreign Application Priority Data
Mar. 21, 2024 (JP) 2024-045627

(51) Int. Cl. A61B 6/03; A61B 5/00

(52) U.S. Cl. CPC A61B 6/03; A61B 5/0033

(57) ABSTRACT

An image processing device includes a processor, in which the processor is configured to: derive, for each of a plurality of two-dimensional images including a structure and having a spatial connection, existence range information for defining a spatial existence range of the structure in a direction intersecting the two-dimensional images, by using a derivation model; and integrate the existence range information derived for each of the plurality of two-dimensional images, based on spatial positions of the plurality of two-dimensional images, by using an integration model.

### CROSS REFERENCE TO RELATED APPLICATIONS

The present application claims priority from Japanese Patent Application No. 2024-045627, filed on Mar. 21, 2024, the entire disclosure of which is incorporated herein by reference.

### BACKGROUND

### Technical Field

The present disclosure relates to an image processing device, an image processing method, and an image processing program.

### Related Art

In recent years, with the advancement of medical equipment, such as a computed tomography (CT) apparatus and a magnetic resonance imaging (MRI) apparatus, three-dimensional images having a higher quality and a higher resolution have been used for image diagnosis.

In a case in which a subject is imaged by using an imaging apparatus, such as the CT apparatus or the MRI apparatus, in order to determine an imaging range, scout imaging is performed before main imaging for acquiring a three-dimensional image to acquire a two-dimensional image for positioning (scout image). An operator of an imaging apparatus, such as a technician, sets the imaging range at the time of main imaging while viewing the scout image.

Meanwhile, the setting of the imaging range while viewing the scout image requires time because the operator needs to perform the setting manually. In addition, since the setting accuracy depends on the ability and the experience of the operator, there is a variation in the setting accuracy. Therefore, a method of estimating a three-dimensional position of an organ included in a tomographic image constituting a scout image has been proposed (see, for example, WO2021/205990A).

By using the method described in WO2021/205990A, it is possible to derive a bounding box for defining the three-dimensional position of the organ included in the tomographic image, for each tomographic image, to integrate position coordinates by obtaining a statistical value of position coordinates of the bounding box derived for each tomographic image, and to obtain a bounding box for specifying a most probable three-dimensional position of the organ. However, since the method described in WO2021/205990A does not consider the position of the tomographic image, there is a possibility that a coordinate position of the integrated bounding box is not accurate.

### SUMMARY OF THE INVENTION

The present disclosure has been made in view of the above-described circumstances, and an object of the present disclosure is to enable highly accurate derivation of a position of a structure such as an organ in consideration of a position of a tomographic image.

The present disclosure provides an image processing device comprising: a processor, in which the processor is configured to: derive, for each of a plurality of two-dimensional images including a structure and having a spatial connection, existence range information for defining a spatial existence range of the structure in a direction intersecting the two-dimensional images, by using a derivation model; and integrate the existence range information derived for each of the plurality of two-dimensional images, based on spatial positions of the plurality of two-dimensional images, by using an integration model.

The present disclosure provides an image processing method executed by a computer, the image processing method including: deriving, for each of a plurality of two-dimensional images including a structure and having a spatial connection, existence range information for defining a spatial existence range of the structure in a direction intersecting the two-dimensional images, by using a derivation model; and integrating the existence range information derived for each of the plurality of two-dimensional images, based on spatial positions of the plurality of two-dimensional images, by using an integration model.

The present disclosure provides an image processing program causing a computer to execute a procedure including: deriving, for each of a plurality of two-dimensional images including a structure and having a spatial connection, existence range information for defining a spatial existence range of the structure in a direction intersecting the two-dimensional images, by using a derivation model; and integrating the existence range information derived for each of the plurality of two-dimensional images, based on spatial positions of the plurality of two-dimensional images, by using an integration model.

According to the present disclosure, the position of the structure can be derived with high accuracy in consideration of the position of the tomographic image.

### BRIEF DESCRIPTION OF THE DRAWINGS

FIG. 1 is a diagram showing a schematic configuration of a medical information system to which an image processing device according to an embodiment of the present disclosure is applied.

FIG. 2 is a diagram showing a schematic configuration of the image processing device according to the present embodiment.

FIG. 3 is a functional configuration diagram of the image processing device according to the present embodiment.

FIG. 4 is a diagram showing processing performed by a derivation unit and an integration unit.

FIG. 5 is a diagram showing a range in which a liver exists.

FIG. 6 is a diagram showing training data.

FIG. 7 is a diagram showing learning.

FIG. 8 is a diagram showing a display screen of a bounding box.

FIG. 9 is a flowchart showing processing performed in the present embodiment.

FIG. 10 is a diagram showing another processing performed by the derivation unit and the integration unit.

### DETAILED DESCRIPTION

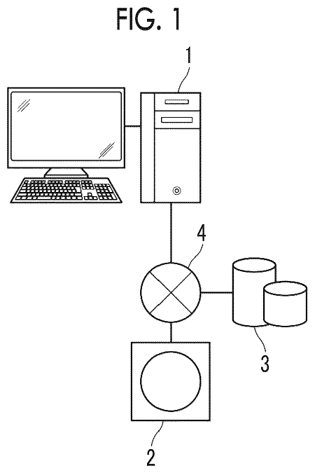

Hereinafter, an embodiment of the present disclosure will be described with reference to the drawings. First, a configuration of a medical information system to which an image processing device according to the present embodiment is applied will be described. FIG. 1 is a diagram showing a schematic configuration of the medical information system. In the medical information system shown in FIG. 1, a computer 1 including the image processing device according to the present embodiment, an imaging apparatus 2, and an image storage server 3 are connected via a network 4 in a communicable state.

The computer 1 includes the image processing device according to the present embodiment, and an image processing program according to the present embodiment is installed in the computer 1. The computer 1 may be a workstation or a personal computer directly operated by a doctor who makes a diagnosis, or may be a server computer connected to the workstation or the personal computer via the network. The image processing program is stored in a storage device of the server computer connected to the network or in a network storage to be accessible from the outside, and is, in response to a request, downloaded and installed in the computer 1 used by the doctor. Alternatively, the image processing program is distributed in a state of being recorded on a recording medium, such as a digital versatile disc (DVD) or a compact disc read-only memory (CD-ROM), and is installed in the computer 1 from the recording medium.

The imaging apparatus 2 is an apparatus that generates a two-dimensional image or a three-dimensional image representing a part of a subject to be diagnosed by imaging the part, and is specifically a radiography apparatus, a computed tomography (CT) apparatus, a magnetic resonance imaging (MRI) apparatus, a positron emission tomography (PET) apparatus, or the like. The image of the subject generated by the imaging apparatus 2 is transmitted to the image storage server 3 and stored in the image storage server 3. It should be noted that the three-dimensional image includes a plurality of tomographic images or an image composed of three-dimensional coordinates generated from the plurality of tomographic images.

The image storage server 3 is a computer that stores and manages various types of data, and comprises a large-capacity external storage device and software for database management. The image storage server 3 communicates with another device via the wired or wireless network 4, and transmits and receives image data and the like to and from the other device. Specifically, the image storage server 3 acquires various types of data including the image data of the image generated by the imaging apparatus 2 via the network, and stores and manages the various types of data in the recording medium, such as the large-capacity external storage device. It should be noted that a storage format of the image data and the communication between the devices via the network 4 are based on a protocol such as digital imaging and communication in medicine (DICOM).

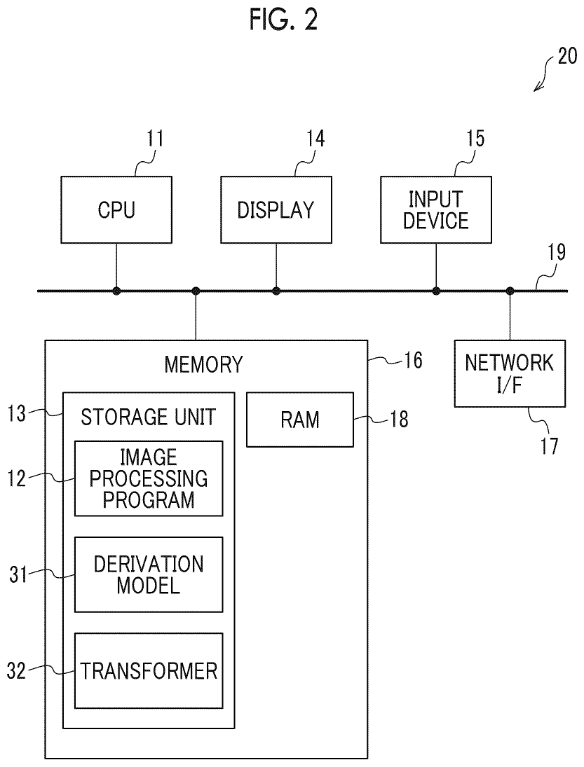

Hereinafter, the image processing device according to the present embodiment will be described. FIG. 2 is a diagram showing a hardware configuration of the image processing device according to the present embodiment. As shown in FIG. 2, the image processing device 20 includes a central processing unit (CPU) 11, a display 14, an input device 15, a memory 16, and a network interface (I/F) 17 connected to the network 4. The CPU 11, the display 14, the input device 15, the memory 16, and the network I/F 17 are connected to a bus 19. It should be noted that the CPU 11 is an example of a processor in the present disclosure.

The memory 16 includes the storage unit 13 and a random access memory (RAM) 18. The RAM 18 is a primary storage memory, and is, for example, a RAM such as a static random access memory (SRAM) or a dynamic random access memory (DRAM).

The storage unit 13 is a non-volatile memory and is implemented by, for example, at least one of a hard disk drive (HDD), a solid state drive (SSD), an electrically erasable and programmable read only memory (EEPROM), or a flash memory. An image processing program according to the present embodiment is stored in the storage unit 13 as a storage medium. The CPU 11 reads out the image processing program 12 from the storage unit 13, loads the readout image processing program 12 in the RAM 18, and executes the loaded image processing program 12. It should be noted that the storage unit 13 also stores a derivation model 31 and a transformer 32, which will be described below.

The display 14 is a device that displays various screens, and is, for example, a liquid crystal display or an electro luminescence (EL) display. The input device 15 is a device for a user to perform input, and is, for example, at least any one of a keyboard, a mouse, a microphone for audio input, a touchpad for proximity input including contact, or a camera for gesture input. The network I/F 17 is an interface for connection to the network 4.

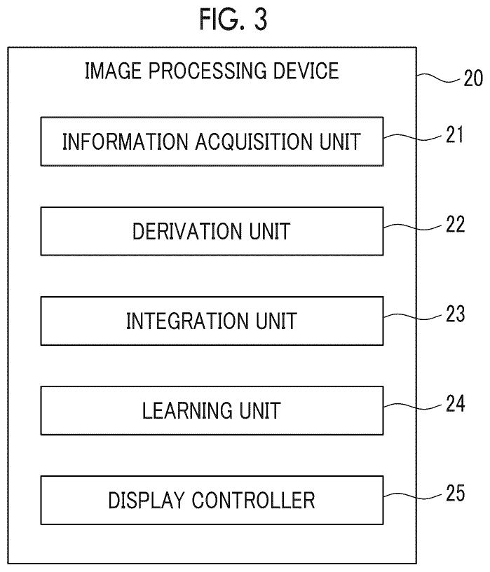

Hereinafter, a functional configuration of the image processing device according to the present embodiment will be described. FIG. 3 is a diagram showing the functional configuration of the image processing device according to the present embodiment. As shown in FIG. 3, the image processing device 20 comprises an information acquisition unit 21, a derivation unit 22, an integration unit 23, a learning unit 24, and a display controller 25. In a case in which the CPU 11 executes the image processing program, the CPU 11 functions as the information acquisition unit 21, the derivation unit 22, the integration unit 23, the learning unit 24, and the display controller 25.

The information acquisition unit 21 acquires a medical image G0 that is a processing target from the image storage server 3 in response to an instruction issued from an operator by using the input device 15. In the present embodiment, the medical image G0 is a scout image used for positioning during the imaging using the CT apparatus or during the imaging using the MRI apparatus. The scout image includes a plurality of tomographic images, has a larger slice interval than the three-dimensional image, and has a smaller number of tomographic images than the three-dimensional image. Therefore, in the present embodiment, the three-dimensional image is referred to as a thin slice image and an image having a large slice interval, such as the scout image, is referred to as a thick slice image.

A difference between the thin slice image and the thick slice image is a difference in resolution in a direction perpendicular to a slice plane. Since the slices are dense in a direction perpendicular to the slice plane in the thin slice image, a structure can be recognized with high accuracy. Meanwhile, since the slice interval in a direction perpendicular to the slice plane is larger in the thick slice image than in the thin slice image, the accuracy of reproducing the structure is lower in the thick slice image than in the thin slice image. It should be noted that, in the present embodiment, the scout image is an image having a larger slice interval than the three-dimensional image, but the present disclosure is not limited to this. Since the slice image need only have a resolution in a direction perpendicular to the slice plane smaller than that of the three-dimensional image, the slice image includes an image having a slice thickness larger than that of the three-dimensional image.

It should be noted that, in the present embodiment, it is assumed that the scout image for acquiring a three-dimensional image of a liver is acquired as the medical image G0 by the imaging apparatus 2.

In addition, the information acquisition unit 21 acquires training data used to train a derivation model and an integration model, which will be described below, from the image storage server 3. The training data and the learning will be described below.

The derivation unit 22 derives, for each of a plurality of two-dimensional images including the structure and having a spatial connection, existence range information for defining a spatial existence range of the structure in a direction intersecting the two-dimensional images, by using the derivation model. That is, the derivation unit 22 uses the derivation model 31 to define the existence range of the structure in a plane of the tomographic image and to derive, as the existence range information, the three-dimensional coordinates for defining the positions of the upper and lower end parts of the liver outside the tomographic plane in a direction intersecting the tomographic image for each of the plurality of tomographic images included in the medical image G0. The existence range information is, for example, three-dimensional coordinates of a plurality of vertices for defining a rectangular parallelepiped surrounding the structure in the three-dimensional space, that is, a bounding box. In particular, in the present embodiment, the derivation unit 22 derives, as the existence range information, the three-dimensional coordinates of two vertices (diagonal vertices) farthest from each other among the plurality of vertices for defining the bounding box.

The integration unit 23 integrates the existence range information derived for each of the plurality of two-dimensional images, by using the transformer 32, based on the spatial positions of the plurality of two-dimensional images. That is, the three-dimensional coordinates of the diagonal vertices of the bounding box derived for each of the plurality of tomographic images included in the medical image G0 are subjected to positional encoding and input to the transformer 32, to derive the integrated three-dimensional coordinates of the diagonal vertices. The transformer 32 is an example of an integration model according to the present disclosure.

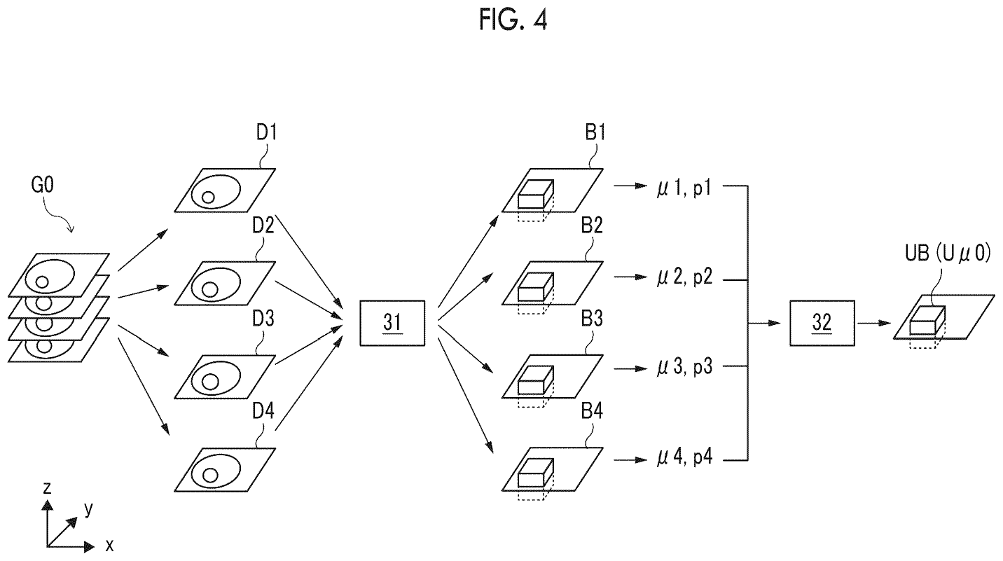

FIG. 4 is a diagram showing processing performed by the derivation unit 22 and the integration unit 23. It should be noted that, in FIG. 4, it is assumed that the medical image G0 includes four tomographic images D1 to D4 of axial cross sections. The tomographic images D1 to D4 are examples of a plurality of tomographic images having a same imaging direction according to the present disclosure. As shown in FIG. 4, the orthogonal directions in the plane of the tomographic images D1 to D4 are an x-direction and a y-direction, and the direction intersecting the tomographic images D1 to D4 is a z-direction.

The derivation unit 22 derives, as the existence range information, three-dimensional coordinates of diagonal vertices of bounding boxes B1 to B4 for defining the spatial existence range of the liver included in the tomographic images D1 to D4, respectively, based on the tomographic images D1 to D4 by using the derivation model 31.

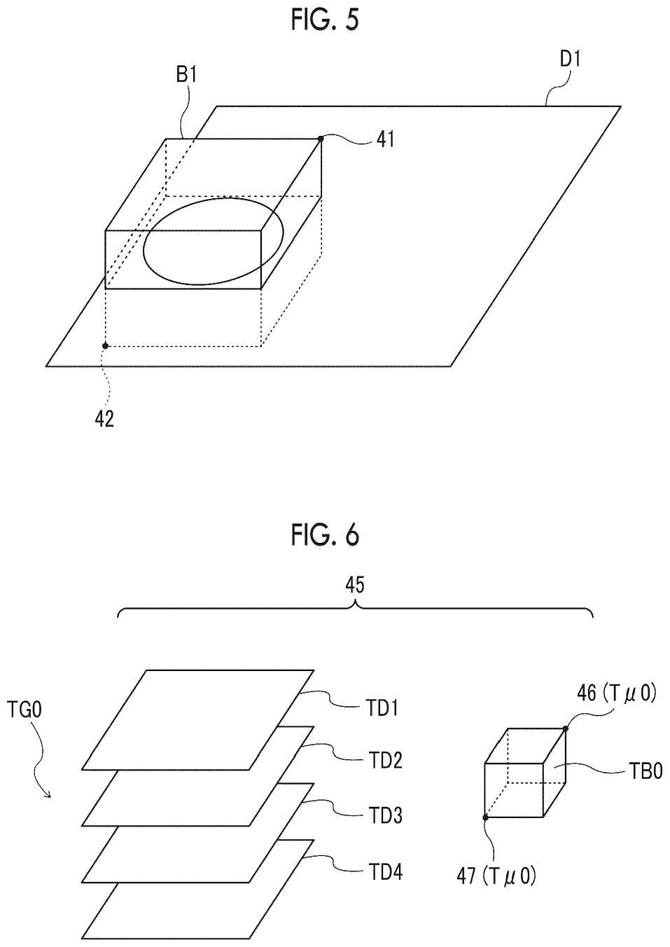

Here, the bounding boxes B1 to B4 are rectangular parallelepipeds having sides parallel to the x-direction, the y-direction, and the z-direction. In a case in which the diagonal vertex is defined among the eight vertices for defining the bounding boxes B1 to B4, a shape of the rectangular parallelepiped can be defined. For example, in a case in which two vertices 41 and 42 that are end points of a diagonal line are defined for the bounding box B1 of the tomographic image D1 shown in FIG. 5, a rectangular parallelepiped shape of the bounding box B1 can be defined. Therefore, in the present embodiment, the derivation model 31 outputs the three-dimensional coordinates of the diagonal vertex among the eight vertices for defining the bounding box as the existence range information.

The derivation model 31 is constructed by, for example, a method described in WO2021/205990A. That is, the derivation model 31 consists of, for example, a convolutional neural network (hereinafter, a CNN), and is constructed by performing machine learning using training data so that, in a case in which the tomographic image is input, a position of the structure included in the input tomographic image within the tomographic plane is defined, and three-dimensional coordinate information for defining the position outside the tomographic plane of the end part of the structure in a direction intersecting the tomographic image is output. The machine learning for constructing the derivation model 31 will be described below.

In the present embodiment, the derivation model 31 is the three-dimensional coordinates of the two diagonal vertices of the bounding box. Therefore, the existence range information is a six-dimensional feature vector. The existence range information derived for each of the tomographic images D1 to D4 is denoted by μ1 to μ4.

The transformer 32 is constructed by using a transformer model. The transformer 32 is proposed, for example, in “Vaswani, Ashish, et al. “Attention is all you need.” Advances in neural information processing systems. 2017”. The transformer 32 integrates the feature vectors by repeating processing of deriving a degree of similarity between the input feature vectors and adding the feature vectors in accordance with a weight corresponding to the derived degree of similarity, and outputs the integrated feature vectors.

In the present embodiment, in a case in which pieces of the existence range information μ1 to μ4 are input to the transformer 32, positional encoding is performed to incorporate the position information in the z-direction of the tomographic image into each existence range information (that is, the feature vector). In the present embodiment, pieces of position information p1 to p4 of the tomographic images D1 to D4 in the z-direction are incorporated into the existence range information μ1 to μ4, respectively. The position information of the tomographic images D1 to D4 in the z-direction can be represented by a one-dimensional vector only in the z-direction. Therefore, the feature vector input to the transformer 32 has seven dimensions together with the feature value vector of the diagonal vertex.

As a reference point of the positional encoding, for example, in the present embodiment, the centroids of the bounding boxes B1 to B4 derived for the tomographic images D1 to D4 can be derived, and a representative value of the z-coordinates of the derived centroids can be used. As the representative value, an average value and a median value of the z-coordinates of the centroids can be used, but the present disclosure is not limited to this. Alternatively, the centroid of any of the bounding boxes B1 to B4 may be derived as the reference point, and the z-coordinate of the center of the surface intersecting the bounding box in the tomographic image at a position closest to the z-coordinate of the centroid may be used.

In the present embodiment, pieces of the positional-encoded existence range information μ1 to μ4 are input to the transformer 32, the existence range information is integrated, and the integrated existence range information, that is, the three-dimensional coordinates of the diagonal vertex of an integrated bounding box UB are output from the transformer 32 as integrated existence range information Uμ0.

It should be noted that, in a case in which the feature vector is input to the transformer 32, the positional encoding may be performed by weighting the feature vector. For example, the positional encoding may be performed by increasing the weighting for the existence range information derived from the tomographic image located closer to the center among the tomographic images from which the existence range information is derived, and decreasing the weighting for the existence range information derived from the tomographic image located farther from the center.

In addition, the weighting may be increased for the existence range information derived from the tomographic image having a larger area of the included structure (that is, the liver) among the plurality of tomographic images. In addition, the weighting may be increased for the existence range information derived from the tomographic image including the structure having a higher anatomical relevance to the structure to be diagnosed among the structures included in the tomographic image. For example, in a case in which the structure is the liver, the weighting in a case of performing the positional encoding may be increased for the existence range information derived from the tomographic image including a blood vessel related to the liver, a part of the intestine, or the like.

The learning unit 24 constructs the derivation model 31 and the transformer 32 through the machine learning. The derivation model 31 is constructed by training a convolutional neural network (CNN), and the transformer 32 is constructed by training a transformer model (TFM). For training the CNN and the TFM, the tomographic image and the three-dimensional coordinates of the diagonal vertices of the bounding box for the structure included in the tomographic image are used as the training data.

FIG. 6 is a diagram showing the training data. As shown in FIG. 6, training data 45 consists of a medical image for training TG0 including a plurality of tomographic images for training TD1 to TD4 and three-dimensional coordinate information Tμ0 of a bounding box for training TB0 representing the existence range of the structure (liver) included in the medical image for training TG0. The three-dimensional coordinate information Tμ0 is three-dimensional coordinates of diagonal vertices 46 and 47 of the bounding box for training TB0. The three-dimensional coordinate information Tμ0 is ground truth data in the training data.

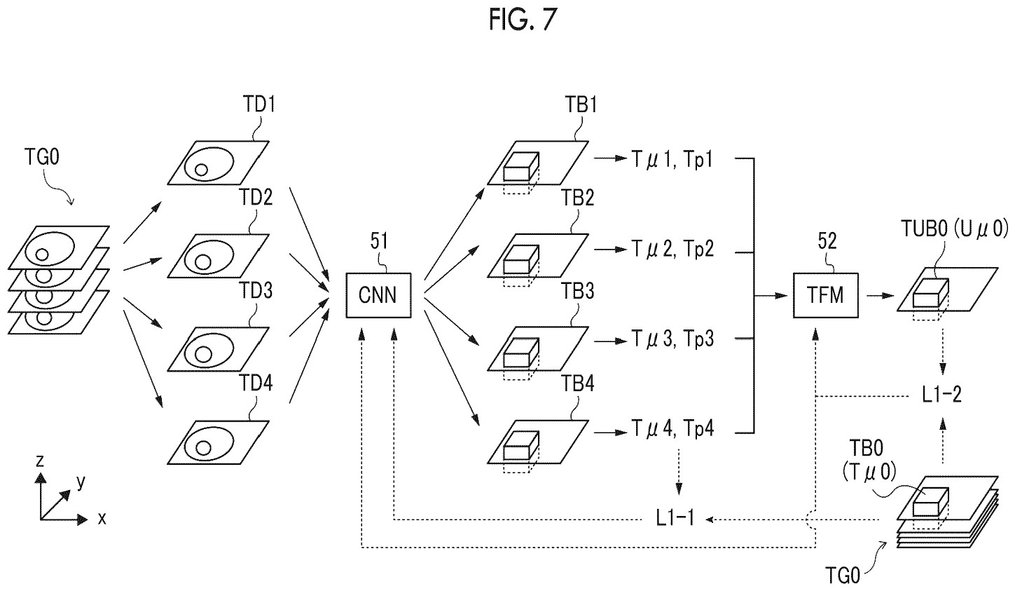

FIG. 7 is a diagram showing training of the CNN and the TFM. The learning unit 24 inputs the tomographic images for training TD1 to TD4 included in the medical image for training TG0 to a CNN 51 and causes the CNN 51 to output the bounding boxes for training TB1 to TB4 for defining the existence ranges of the structure included in the tomographic images for training TD1 to TD4. Specifically, the three-dimensional coordinates of the diagonal vertices of the bounding boxes for training TB1 to TB4 are output as existence range information for training Tμ1 to Tμ4.

The learning unit 24 performs the positional encoding on the existence range information for training Tμ1 to Tμ4 to incorporate the position information Tp1 to Tp4 in the z-direction of the tomographic images for training TD1 to TD4 into each of the existence range information for training Tμ1 to Tμ4. Then, the learning unit 24 inputs the positional-encoded existence range information for training Tμ1 to Tμ4 to a TFM 52, integrates the existence range information for training Tμ1 to Tμ4, and outputs the three-dimensional coordinates of the diagonal vertex of the integrated bounding box TUBO from the TFM 52 as the integrated existence range information Uμ0.

The learning unit 24 derives a difference between the existence range information for training Tμ1 to Tμ4 and the three-dimensional coordinate information Tμ0 of the diagonal coordinates of the bounding box for training TB0 of the training data 45, which is the ground truth data, as a first loss L1-1. In addition, the learning unit 24 derives a difference between the integrated existence range information Uμ0 and the three-dimensional coordinate information Tμ0 of the training data 45 as a second loss L1-2. Then, the learning unit 24 trains the CNN 51 by performing, as appropriate, weighting on the first loss L1-1 and the second loss L1-2 so that the first loss L1-1 and the second loss L1-2 are equal to or less than a predetermined threshold value. In addition, the learning unit 24 trains the TFM 52 by performing, as appropriate, weighting on the second loss L1-2 so that the second loss L1-2 is equal to or less than the predetermined threshold value.

As the learning progresses, in a case in which each of the plurality of tomographic images included in the medical image is input, the CNN 51 can accurately derive the three-dimensional coordinates of the diagonal vertex of v bounding box for defining the spatial existence range of the structure included in the tomographic image. In addition, in a case in which the existence range information (that is, the three-dimensional coordinates of the diagonal vertex of the bounding box) of the structure in the plurality of positional-encoded tomographic images is input, the TFM 52 can integrate the existence range information and accurately derive the three-dimensional coordinates of the diagonal vertex of the bounding box for defining the spatial existence range of the structure included in the medical image. By advancing the learning in this way, the CNN 51 is constructed as the derivation model 31, and the TFM 52 is constructed as the transformer 32.

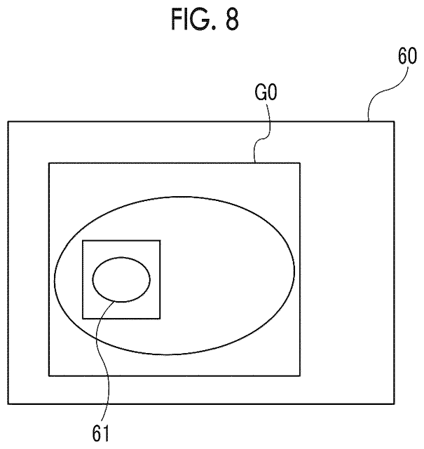

The display controller 25 displays the existence range of the structure derived from the input medical image G0, that is, the bounding box, on the medical image G0 in a superimposed manner. FIG. 8 is a diagram showing a display screen. As shown in FIG. 8, the medical image G0 on which a bounding box 61 is displayed in a superimposed manner is displayed on a display screen 60. It should be noted that the displayed medical image G0 is one tomographic image among the tomographic images included in the medical image G0, and the operator can switch the displayed tomographic image by operating the input device 15.

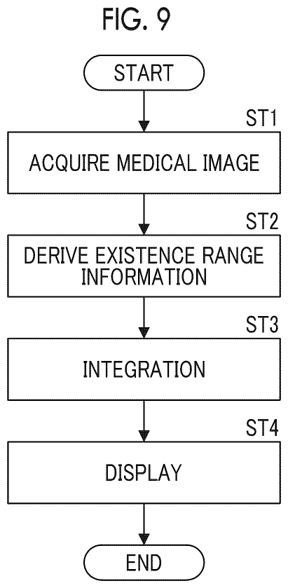

Hereinafter, the processing performed in the present embodiment will be described. FIG. 9 is a flowchart showing processing performed in the present embodiment. First, the information acquisition unit 21 acquires the medical image G0 as a processing target from the image storage server 3 (step ST1). Next, the derivation unit 22 derives the existence range information indicating the spatial existence range of the structure for each of the plurality of tomographic images D1 to D4 included in the medical image G0 (step ST2). Next, the integration unit 23 integrates the existence range information derived for the plurality of tomographic images D1 to D4 to derive the integrated existence range information (step ST3). Next, the display controller 25 displays the medical image G0 on the display 14 by superimposing the bounding box based on the integrated existence range information (step ST4), and the processing ends.

As described above, in the present embodiment, for each of the plurality of tomographic images included in the medical image G0, the existence range information for defining the spatial existence range of the structure in a direction intersecting the tomographic image is derived by using the derivation model 31, and the existence range information is integrated by using the transformer 32 based on the spatial positions of the plurality of tomographic images D1 to D4. Therefore, it is possible to accurately define the position of the structure in consideration of the position of the tomographic image.

It should be noted that, in the above-described embodiment, the existence range information indicating the existence range of the structure is derived from the plurality of tomographic images of the axial cross section included in the medical image G0, but the present disclosure is not limited to this. The existence range information indicating the existence range of the structure may be derived by using the medical image G0 including the tomographic images of a sagittal cross section and a coronal cross section in addition to the axial cross section.

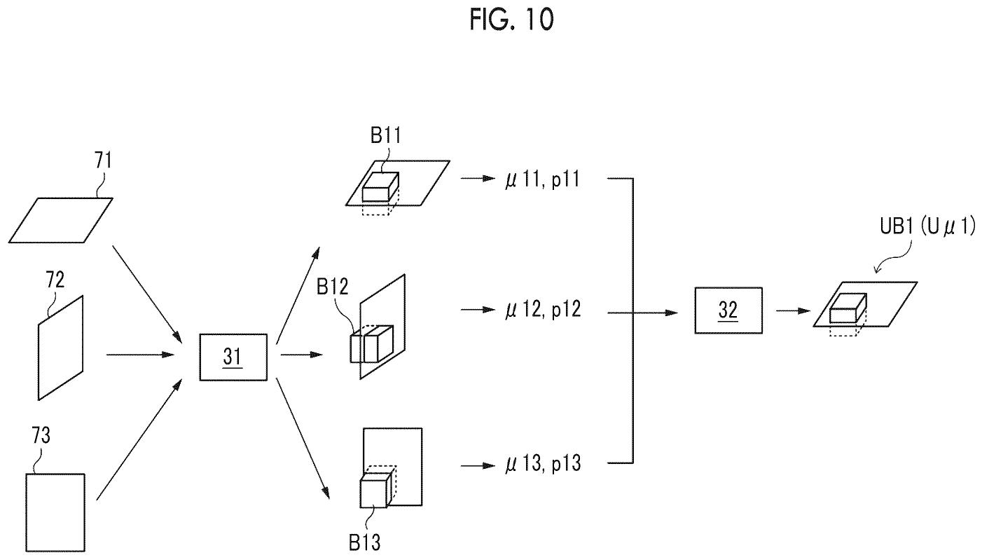

For example, as shown in FIG. 10, bounding boxes B11 to B13, that is, existence range information μ11, μ12, and μ13 may be derived from each of a tomographic image 71 of the axial cross section, a tomographic image 72 of the sagittal cross section, and a tomographic image 73 of the coronal cross section by using the derivation model 31, the positional encoding may be performed to incorporate the position information p11, p12, and p13 of each of the tomographic image 71 of the axial cross section, the tomographic image 72 of the sagittal cross section, and the tomographic image 73 of the coronal cross section, then the existence range information μ11, μ12, and μ13 may be integrated by using the transformer 32, and the three-dimensional coordinates of the diagonal vertex of an integrated bounding box UB1 may be derived as integrated existence range information Uμ1. The tomographic images 71 to 73 are examples of tomographic images having different imaging directions and obtained for a plurality of tomographic planes that share three-dimensional absolute coordinate values according to the present disclosure.

In this case, the position information p11 to p13 of the tomographic plane in a case of performing the positional encoding is three-dimensional information, not one-dimensional information. Therefore, since the three-dimensional position information is incorporated into the existence range information, which is the six-dimensional feature vector, by the positional encoding, a nine-dimensional feature vector is input to the transformer 32, and the integrated existence range information Uμ1 is derived.

In addition, in the above-described embodiment, the derivation unit 22 derives the three-dimensional coordinates for defining the diagonal vertex of the bounding box in the tomographic images D1 to D4 as the existence range information, but the present disclosure is not limited to this. The feature values for specifying the three-dimensional coordinates, which is derived in the derivation model 31 at a stage of calculation, may be derived. In this case, the integration unit 23 integrates the feature values by using the transformer 32 to derive the integrated existence range information of the structure included in the medical image G0.

In addition, in the above-described embodiment, the scout image is the processing target, but the present disclosure is not limited to this. The range of the structure may be derived by using, as a target, the thin slice image included in the three-dimensional image acquired by the main imaging.

In addition, in the above-described embodiment, the structure is an organ such as the liver, but the present disclosure is not limited to this. At least one of a lesion, a high signal region in the image, or a high density region in the image may be used as the structure to derive the range thereof.

In this embodiment, each process is executed on an arbitrary computer. The arbitrary computer may execute these processes by means of a processor as hardware, a program as software, or a combination of the processor and the program. In such a case, the processor is configured to execute the various processes in this embodiment in cooperation with the program and may function as each unit or means in this embodiment. In addition, the order in which the processor executes these processes is not limited to the order described in this embodiment and may be changed as appropriate. The arbitrary computer may be a general-purpose computer, a computer for a specific purpose, a workstation, or any other system capable of executing each process.

The processor may be configured by one or more hardware, and the type of hardware is not limited. For example, the processor may comprise at least one of programmable logic devices such as CPUs (Central Processing Units), MPUs (Micro Processing Units), and FPGAs (Field Programmable Gate Arrays); dedicated circuits for performing specific processes such as ASICs (Application Specific Integrated Circuits); and other hardware such as a GPU (Graphics Processing Unit) and an NPU (Neural Processing Unit). The hardware may also be a combination of different types of hardware. When multiple hardware are configured to execute one or more processes of a processor, the said multiple hardware may exist in devices that are physically separate from each other, or in the same device. In any embodiment, the order of each process by the processor is not limited to the order described above and may be changed as appropriate. The hardware is configured by an electric circuit (circuitry) etc. that combines circuit elements such as semiconductor devices.

Furthermore, the program may be firmware or software such as microcode. The program may also be a group of program modules, each function of which may be performed by a processor configured to execute each of the program modules. The program may be program code or code segments stored on one or more non-transitory computer-readable media (e.g., storage media or other storage). The program may be stored in separate non-transitory computer-readable media located on devices that are physically separate from each other. The program code or code segments may represent any combination of procedures, functions, subprograms, routines, subroutines, modules, software packages, classes, instructions, data structures, or program statements. The program code or code segments may be connected to other code segments or hardware circuits by sending or receiving information, data, arguments, parameters, or memory contents.

In the above embodiment, it is explained that the image processing program 12 is stored (installed) in advance in the storage section 13, but this is not limited to this. The image processing program 12 may be provided in a form recorded on a recording medium such as a CD-ROM (Compact Disc Read Only Memory), DVD-ROM (Digital Versatile Disc Read Only Memory), and USB (Universal Serial Bus) memory. In addition, the image processing program 12 may be provided in a form that the image processing program 12 is downloaded from an external device via a network.

The technology of this disclosure also extends to all types of program products. Program products include all types of products for providing programs. For example, program products include programs provided via networks such as the Internet, and non-temporary computer readable storage media such as CD-ROMs, DVDs, and USB memory devices that store programs.

Hereinafter, the supplementary notes of the present disclosure will be described.

### Supplementary Note 1

An image processing device comprising: a processor, in which the processor is configured to: derive, for each of a plurality of two-dimensional images including a structure and having a spatial connection, existence range information for defining a spatial existence range of the structure in a direction intersecting the two-dimensional images, by using a derivation model; and integrate the existence range information derived for each of the plurality of two-dimensional images, based on spatial positions of the plurality of two-dimensional images, by using an integration model.

### Supplementary Note 2

The image processing device according to supplementary note 1, in which the plurality of two-dimensional images having the spatial connection are a plurality of tomographic images having a same imaging direction.

### Supplementary Note 3

The image processing device according to supplementary note 1, in which the plurality of two-dimensional images having the spatial connection are tomographic images having different imaging directions and obtained for a plurality of tomographic planes that share three-dimensional absolute coordinate values.

### Supplementary Note 4

The image processing device according to any one of supplementary notes 1 to 3, in which the processor is configured to: derive three-dimensional coordinates for defining the existence range of the structure or feature values for specifying the three-dimensional coordinates, as the existence range information, by using the derivation model; and integrate the three-dimensional coordinates or the feature values derived for each of the plurality of two-dimensional images, by using the integration model.

### Supplementary Note 5

The image processing device according to any one of supplementary notes 1 to 4, in which the existence range information is three-dimensional coordinates of two vertices located at farthest positions among a plurality of vertices for defining a rectangular parallelepiped surrounding the structure.

### Supplementary Note 6

The image processing device according to any one of supplementary notes 1 to 5, in which the processor is configured to: perform weighting on the existence range information in accordance with a manner in which the structure is included in the two-dimensional image; and integrate the weighted existence range information by using the integration model.

### Supplementary Note 7

The image processing device according to supplementary note 6, in which the processor is configured to: increase the weighting as the existence range of the structure included in the two-dimensional image becomes larger.

### Supplementary Note 8

The image processing device according to supplementary note 6, in which the processor is configured to: increase the weighting as an anatomical connection of the existence range becomes stronger in the plurality of two-dimensional images.

### Supplementary Note 9

The image processing device according to any one of supplementary notes 1 to 8, in which the processor is configured to: derive a representative value based on the existence range information derived for each of the plurality of two-dimensional images, as a reference point for positions of the plurality of two-dimensional images.

### Supplementary Note 10

The image processing device according to supplementary note 9, in which the reference point is a representative value of a center position of the existence range of the structure derived based on the existence range information, for the plurality of two-dimensional images.

### Supplementary Note 11

The image processing device according to supplementary note 9, in which the reference point is a center position in a region in which the existence range of the structure defined by the existence range information derived for a selected two-dimensional image among the plurality of two-dimensional images intersects the selected two-dimensional image.

### Supplementary Note 12

An image processing method executed by a computer, the image processing method including: deriving, for each of a plurality of two-dimensional images including a structure and having a spatial connection, existence range information for defining a spatial existence range of the structure in a direction intersecting the two-dimensional images, by using a derivation model; and integrating the existence range information derived for each of the plurality of two-dimensional images, based on spatial positions of the plurality of two-dimensional images, by using an integration model.

### Supplementary Note 13

An image processing program causing a computer to execute a procedure including: deriving, for each of a plurality of two-dimensional images including a structure and having a spatial connection, existence range information for defining a spatial existence range of the structure in a direction intersecting the two-dimensional images, by using a derivation model; and integrating the existence range information derived for each of the plurality of two-dimensional images, based on spatial positions of the plurality of two-dimensional images, by using an integration model.

What is claimed is:

1. An image processing device comprising:
a processor,
wherein the processor is configured to:
derive, for each of a plurality of two-dimensional images including a structure and having a spatial connection, existence range information for defining a spatial existence range of the structure in a direction intersecting the two-dimensional images, by using a derivation model; and
integrate the existence range information derived for each of the plurality of two-dimensional images, based on spatial positions of the plurality of two-dimensional images, by using an integration model.

2. The image processing device according to claim 1,
wherein the plurality of two-dimensional images having the spatial connection are a plurality of tomographic images having a same imaging direction.

3. The image processing device according to claim 1,
wherein the plurality of two-dimensional images having the spatial connection are tomographic images having different imaging directions and obtained for a plurality of tomographic planes that share three-dimensional absolute coordinate values.

4. The image processing device according to claim 1,
wherein the processor is configured to:
derive three-dimensional coordinates for defining the existence range of the structure or feature values for specifying the three-dimensional coordinates, as the existence range information, by using the derivation model; and
integrate the three-dimensional coordinates or the feature values derived for each of the plurality of two-dimensional images, by using the integration model.

5. The image processing device according to claim 1,
wherein the existence range information is three-dimensional coordinates of two vertices located at farthest positions among a plurality of vertices for defining a rectangular parallelepiped surrounding the structure.

6. The image processing device according to claim 1,
wherein the processor is configured to:
perform weighting on the existence range information in accordance with a manner in which the structure is included in the two-dimensional image; and
integrate the weighted existence range information by using the integration model.

7. The image processing device according to claim 6,
wherein the processor is configured to:
increase the weighting as the existence range of the structure included in the two-dimensional image becomes larger.

8. The image processing device according to claim 6,
wherein the processor is configured to:
increase the weighting as an anatomical connection of the existence range becomes stronger in the plurality of two-dimensional images.

9. The image processing device according to claim 1,
wherein the processor is configured to:
derive a representative value based on the existence range information derived for each of the plurality of two-dimensional images, as a reference point for positions of the plurality of two-dimensional images.

10. The image processing device according to claim 9,
wherein the reference point is a representative value of a center position of the existence range of the structure derived based on the existence range information, for the plurality of two-dimensional images.

11. The image processing device according to claim 9,
wherein the reference point is a center position in a region in which the existence range of the structure defined by the existence range information derived for a selected two-dimensional image among the plurality of two-dimensional images intersects the selected two-dimensional image.

12. An image processing method executed by a computer, the image processing method comprising:
deriving, for each of a plurality of two-dimensional images including a structure and having a spatial connection, existence range information for defining a spatial existence range of the structure in a direction intersecting the two-dimensional images, by using a derivation model; and
integrating the existence range information derived for each of the plurality of two-dimensional images, based on spatial positions of the plurality of two-dimensional images, by using an integration model.

13. A non-transitory computer-readable storage medium that stores an image processing program causing a computer to execute a procedure comprising:
deriving, for each of a plurality of two-dimensional images including a structure and having a spatial connection, existence range information for defining a spatial existence range of the structure in a direction intersecting the two-dimensional images, by using a derivation model; and
integrating the existence range information derived for each of the plurality of two-dimensional images, based on spatial positions of the plurality of two-dimensional images, by using an integration model.
